(19) United States
(12) Patent Application Publication
(10) Pub. No.: US 2003/0125584 A1
(43) Pub. Date: Jul. 3, 2003

(54) 6-Alkylindan-1-ones as fragrances

(76) Inventors: Steffen Sonnenberg, Holzminden (DE); Oskar Koch, Gottingen (DE); Bertrand Duchaufour, Paris (FR)

Correspondence Address: BAYER POLYMERS LLC, 100 BAYER ROAD, PITTSBURGH, PA, 15205, US

(21) Appl. No.: 10/291,348

(22) Filed: Nov. 8, 2002

(30) Foreign Application Priority Data
Nov. 12, 2001 (DE) 10155554.7

(51) Int. Cl. A61K 7/46; C07C 49/617

(52) U.S. Cl. 568/327; 512/15

(57) ABSTRACT

The invention relates to the use of 6-alkylindan-1-ones as fragrances, to a process for their preparation, to fragrance mixtures comprising 6-alkylindan-1-ones, and to perfumed products comprising 6-alkylindan-1-ones.

### FIELD OF THE INVENTION

The invention relates to the use of 6-alkylindan-1-ones as fragrances, to a process for their preparation, to fragrance mixtures containing 6-alkylindan-1-ones and to perfumed products containing 6-alkylindan-1-ones.

### BACKGROUND OF THE INVENTION

As a result of the generally inadequate availability of natural fragrances and flavors, the constantly expanding and changing demands with regard to perfume and the discontinuation of common fragrances on the basis of toxicological, ecological and cost considerations, there continues to be a need for compounds with valuable fragrance properties. This need applies particularly when the fragrances have additional positive secondary properties, such as, for example, greater stability, higher extendability or better staying power. Of particular interest then are those substances which not only have excellent sensory properties, but also with which, due to their strength and extendability, notable effects can be achieved with low concentrations.

Fragrances with a leather-like and woody odor are increasingly of interest for creating perfumes. The known fragrances with a leather odor mostly belong to the chemical group of phenols or of quinolines. Both chemical groups can generally cause toxicological effects such as skin irritation.

Therefore, there is a need for novel leather fragrances which do not belong to the chemical group of phenols or quinolines and have particular sensory properties.

### SUMMARY OF THE INVENTION

The present invention provides for the use of compounds of the formula (1)

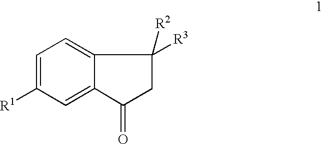

as fragrances, where

R¹ is methyl, ethyl, n-propyl, isopropyl, n-butyl, isobutyl,

R², R³, independently of one another, are H, methyl, ethyl, n-propyl, isopropyl, n-butyl, sec-butyl, isobutyl,

with the proviso that R² and R³ together contain at most 4 carbon atoms and R¹, R² and R³ together contain at most 8 carbon atoms.

The present invention further provides a process for the preparation of the 6-alkylindan-1-ones, and fragrance compositions and perfumed products comprising 6-alkylindan-1-ones of the formula (1).

### DETAILED DESCRIPTION OF THE INVENTION

Preferably, the radicals R¹, R² and R³ have the following meanings:

R¹ is methyl, ethyl, isopropyl

R², R³, independently of one another, are methyl, ethyl.

More preference is given to 3,3,6-trimethylindan-1-one (R¹, R² and R³ are all methyl).

It has been found that the 6-alkylindan-1-ones according to the present invention have a leathery odor with woody notes. The compounds according to the present invention are characterized by an intensive odor, particularly high extendability, high odiferous adhesion and a valuable odor character.

U.S. Pat. No. 4,532,357 describes 3,3,7- and 3,3,5-trimethylindan-1-one as fragrances. It describes the odiferous properties of the mixture of the two isomers, and the two individual isomers.

The sensory properties of the compounds according to the present invention have been investigated with regard to the odor character, the extendability, the intensity and the adhesion compared to the other regio isomers (see Table 1). All of the odor tests were carried out by a group of trained experts with at least six participants with concealed and coded samples.

6-Alkylindan-1-ones are characterized by a very high odiferous extendability, a high odiferous intensity and very high odiferous adhesion. In addition, 6-alkylindan-1-ones are also characterized by a significant leather odor with spicy notes and differ in this respect from the other isomers in which tobacco and honey notes with floral aspects dominate.

To determine the extendability, logarithmically diluted solutions of the compound according to the present invention and of other isomers in diethyl phthalate (DEP) were prepared, and smelling strips were dipped uniformly into these solutions. These smelling strips were then smelled compared with smelling strips which are immersed in pure and odorless diethyl phthalate. In this way, the last dilution to still have an odor was determined by the group of experts. In this connection, it emerged that 3,3,6-trimethylindan-1-one has an extendability which is fifty to five hundred times greater than that of other isomers (Table 1). This property means that even the very smallest amounts of the compounds according to the present invention can be perceived in odor terms and thus can make a considerable contribution in perfume oils and perfumed products.

To determine the odor intensity of the compounds according to the present invention and of the 3,3,4-, 3,3,5- and 3,3,7-trimethyl isomers, the 5% strength by weight ethanolic solutions were smelled from smelling strips. In this connection, the odor intensity was compared on a scale from 1 (weak) to 9 (very strong). In this connection, it is found that, for example, the 3,3,6-trimethylindan-1-one has a significantly higher odor intensity than that of the other isomers (Table 1).

Odor adhesion is the odiferous perception of fragrances over a long period. To evaluate the odor adhesion, the group of experts in each case daily evaluates smelling strips with the compound according to the present invention and the other isomers until a time when no odor, or only a very weak or untypical odor, can be perceived. In this connection, it is found that 3,3,6-trimethylindan-1-one has a considerably longer odor adhesion than the other investigated isomeric trimethylindan-1-ones (Table 1).

TABLE 1
Odor properties of trimethylindan-1-ones

| | | Extendability | | |
|---|---|---|---|---|
| | | [10⁻⁶% | | Adhesion |
| Compound | Odor character | in DEP] | Intensity | [days] |
| 3,3,4-Trimethyl- | Waxy, nutty, | 500 | 6 | 8 |
| indanone | spicy, leathery, | | | |
| | labdanum | | | |
| 3,3,5-Trimethyl- | Green peppero- | 100 | 5 | 9 |
| indanone | ni, isobutyl- | | | |
| | quinoline, | | | |
| | leathery, as- | | | |
| | paragus, green | | | |
| | beans | | | |
| 3,3,6-Trimethyl- | Leathery, | 1 | 7 | 12 |
| indanone | labdanum, | | | |
| | balsamic, | | | |
| | spicy, fruity | | | |
| 3,3,7-Trimethyl- | Honey-like, | 50 | 6 | 8 |
| indanone | woody, bees- | | | |
| | wax, slightly | | | |
| | smoky | | | |

On the basis of the sensory work which has been carried out, it has surprisingly been found that 3,3,6-trimethylindan-1-one has essential advantages over the other isomers.

To assess the individual isomeric trialkylindan-1-ones, they were prepared by reacting alkyl-substituted benzoic acid isobutyl esters in polyphosphoric acid. Thus, 3,3,4-, 3,3,5-, 3,3,6- and 3,3,7-trimethylindan-1-ones, for example, were obtained by reacting para-, meta- or ortho-toluic acid isobutyl esters, respectively, in polyphosphoric acid.

In a mixture with other isomeric trialkylindan-1-ones, such as, for example, 3,3,4-, 3,3,5- or 3,3,7-trimethylindan-1-one, the compounds according to the present invention also develop a pleasant leather-like odor which makes a valuable contribution in perfume compositions. Surprisingly, the compounds according to the present invention in a mixture with isomeric trimethylindan-1-ones impart a particularly intensive odor and produce a particularly high extendability which cannot be achieved by the two isomers alone. This gives rise to considerable advantages when employing the 6-alkylindan-1-ones for use in perfume oils.

The compounds according to the present invention can produce surprising enhancing effects in perfume oils either as an individual fragrance, or as a mixture with isomeric trimethylindan-1-ones or together with other fragrances.

Examples of fragrances in perfume oils can be found, for example, in K. Bauer, D. Garbe and H. Surburg, Common Fragrance and Flavor Materials, 3^{rd}. Ed., Wiley-VCH, Weinheim 1997.

Specifically, mention may be made of:

extracts from natural raw materials such as essential oils, concretes, absolutes, resins, resinoids, balsams, tinctures, such as, for example, ambergris tincture; amyris oil; angelica seed oil; angelica root oil; aniseed oil; valerian oil; basil oil; wood moss absolute; bay oil; mugwort oil; benzoin resin; bergamot oil; beeswax absolute; birch tar oil; bitter almond oil; savory oil; bucco leaf oil; cabreuva oil; cade oil; calmus oil; camphor oil; cananga oil; cardamom oil; cascarilla oil; cassia oil; cassia absolute; castoreum absolute; cedar leaf oil; cedarwood oil; cistus oil; citronella oil; lemon oil; copaiva balsam; copaiva balsam oil; coriander oil; costus root oil; cumin oil; cypress oil; davana oil; dill herb oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; blue camomile oil; Roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil; litsea cubeba oil; bayleaf oil; mace oil; marjoram oil; mandarin oil; massoi bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange-flower absolute; orange oil; origanum oil; palmarosa oil; patchouli oil; perilla oil; Peruvian balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmation sage oil; Spanish sage oil; sandalwood oil; celery seed oil; spike lavender oil; Japanese anise oil; styrax oil; tagetes oil; fir needle oil; tea-tree oil; turpentine oil; thyme oil; Tolu balsam; tonka absolute; tuberose absolute; vanilla extract; violet leaf absolute; verbena oil; vetiver oil; juniper oil; wine lees oil; absinthe oil; wintergreen oil; ylang oil; hyssop oil; civet absolute; cinnamon leaf oil; cinnamon bark oil; and fractions thereof, or ingredients isolated therefrom;

individual fragrances from the group of hydrocarbons, such as, for example, 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene;

of aliphatic alcohols, such as, for example, hexanol; octanol; 3-octanol; 2,6-dimethylheptanol; 2-methylheptanol, 2-methyloctanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol; of aliphatic aldehydes and 1,4-dioxacycloalken-2-ones thereof, such as, for example, hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal diethyl acetal; 1,1-dimethoxy-2,2,5-trimethyl-4-hexene; citronellyl oxyacetaldehyde; of aliphatic ketones and oximes thereof, such as, for example, 2-heptanone; 2-octanone; 3-octanone; 2-nonanone; 5-methyl-3-heptanone; 5-methyl-3-heptanone oxime; 2,4,4,7-tetramethyl-6-octen-3-one; of aliphatic sulfur-containing compounds, such as, for example, 3-methylth iohexanol; 3-methylth iohexyl acetate; 3-mercaptohexanol; 3-mercaptohexyl acetate; 3-mercaptohexyl butyrate; 3-acetylthiohexyl acetate; 1-menthene-8-thiol;

of aliphatic nitriles, such as, for example, 2-nonenenitrile; 2-tridecenenitrile; 2,12-tridecenenitrile; 3,7-dimethyl-2,6-octadienenitrile; 3,7-dimethyl-6-octenenitrile;

of aliphatic carboxylic acids and esters thereof, such as, for example, (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynoate; methyl 2-nonynoate; allyl 2-isoamyloxyacetate; methyl 3,7-dimethyl-2,6-octadienoate;

of acyclic terpene alcohols, such as, for example, citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and formates, acetates, propionates, isobutyrates, butyrates, isovalerates, pentanoates, hexanoates, crotonates, tiglinates, 3-methyl-2-butenoates thereof;

of acyclic terpene aldehydes and ketones, such as, for example, geranial; neral; citronellal; 7-hydroxy-3,7-dimethyloctanal; 7-methoxy-3,7dimethyloctanal; 2,6,10-trimethyl-9-undecenal; geranylacetone; and the dimethyl and diethyl acetals of geranial, neral, 7-hydroxy-3,7-dimethyloctanal;

of cyclic terpene alcohols, such as, for example, menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guaiol; and formates, acetates, propionates, isobutyrates, butyrates, isovalerates, pentanoates, hexanoates, crotonates, tiglinates, 3-methyl-2-butenoates thereof;

of cyclic terpene aldehydes and ketones, such as, for example, menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1 ,3,4,6,7,8a-hexahydro-1,1,5,5-tetramethyl-2H-2,4a-methanonaphthalen-8(5H)-one; nootkatone; dihydronootkatone; alpha-sinensal; beta-sinensal; acetylated cedarwood oil (methyl cedryl ketone);

of cyclic alcohols, such as, for example, 4-tert-butylcyclohexanol; 3,3,5-trimethylcyclohexanol; 3-isocamphylcyclohexanol; 2,6,9-trimethyl-Z2,Z5,E9-cyclo dodecatrien-1-ol; 2-isobutyl-4-methyltetrahydro-2H-pyran-4-ol;

of cycloaliphatic alcohols, such as, for example, alpha-3,3-trimethylcyclohexylmethanol; 2-methyl-4-(2,2,3-trimethyl-3-cyclopent-1-yl)butanol; 2-methyl-4-(2 ,2 ,3-trimethyl-3-cyclopent-1-yl)-2-buten-1-ol; 2-ethyl-4-(2 ,2,3-trimethyl-3-cyclopent-1-yl)-2-buten-1-ol; 3-methyl-5-(2,2,3-trimethyl-3-cyclopent-1-yl )-pentan-2-ol; 3-methyl-5-(2 ,2, 3-trimethyl-3-cyclopent-1-yl )-4-penten-2-ol; 3 ,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;

of cyclic and cycloaliphatic ethers, such as, for example, cineol; cedryl methyl ether; cyclododecyl methyl ether; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-d imethyl-3-cyclohexen-1-yl )-5-methyl-5-(1-methyl propyl)-1,3-dioxane; of cyclic ketones, such as, for example, 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 5-cyclohexadecen-1-one; 8-cyclohexadecen-1-one; 9-cycloheptadecen-1-one; cyclopentadecanone;

of cycloaliphatic aldehydes, such as, for example, 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;

of cycloaliphatic ketones, such as, for example, 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert-butyl 2,4-dimethyl-3-cyclohexen-1-yl ketone;

of esters of cyclic alcohols, such as, for example, 2-tert-butylcyclohexyl acetate; 4-tert-butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl isobutyrate; 4,7-methanooctahydro-5 or 6-indenyl acetate;

of esters of cycloaliphatic carboxylic acids, such as, for example, allyl 3-cyclohexyl-propionate; allyl cyclohexyloxyacetate; methyl dihydrojasmonate; methyl jasmonate; methyl 2-hexyl-3-oxocyclopentanecarboxylate; ethyl 2-ethyl-6,6-dimethyl-2-cyclohexenecarboxylate; ethyl 2,3 ,6,6-tetra methyl-2-cyclohexenecarboxylate; ethyl 2-methyl-1,3-d ioxolan-2-acetate;

of aromatic hydrocarbons, such as, for example, styrene and diphenylmethane;

of araliphatic alcohols, such as, for example, benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

of esters of araliphatic alcohols and aliphatic carboxylic acids, such as, for example, benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate; of araliphatic ethers, such as, for example, 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaldehyde dimethyl acetal; phenylacetaldehyde glycerol acetal; 2,4,6-trimethyl-4-phenyl-1,3-dioxane; 4,4a,5,9b-etrahydroindeno[1,2-d]-m-dioxin; 4,4a,5,9b-tetrahydro-2,4-dimethylindeno[1,2-d]-m-dioxin;

of aromatic and araliphatic aldehydes, such as, for example, benzaldehyde; phenylacetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylened ioxybenzaldehyde; 3,4-dimethoxy-benzaldehyde; 2-methyl-3-(4-methoxyphenyl)propanal; 2-methyl-3-(4-methylenedioxyphenyl)propanal;

of aromatic and araliphatic ketones, such as, for example, acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;

of aromatic and araliphatic carboxylic acids and esters thereof, such as, for example, benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

of nitrogen-containing aromatic compounds, such as, for example, 2,4,6-trinitro-1,3-dimethyl-5-tert-butylbenzene; 3,5-dinitro-2,6-dimethyl-4-tert-butylacetophenone; cinnamonitrile; 5-phenyl-3-methyl-2-pentenenitrile; 5-phenyl-3-methylpentanenitrile; methyl anthranilate; methyl N-methylanthranilate; Schiff bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; indole; skatole; 2-methoxy-3-isopropylpyrazine; 2-isobutyl-3-methoxypyrazine;

of phenols, phenyl ethers and phenyl esters, such as, for example, estragole; anethole; eugenole; eugenyl methyl ether; isoeugenole; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate; of heterocyclic compounds, such as, for example, 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one; of lactones, such as, for example, 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

The preparation of the compounds according to the present invention is described for some compounds. For example, Journal of Am. Chem. Soc. 78, 2622 (1956) describes the preparation of 3,3,6-trimethylindan-1-one. This three-stage synthesis is overall unsatisfactory both with regard to the reaction conditions and also the yield (40% of theory over three stages).

It has now been found that the preparation of 1-indanones is possible in high yields by reacting (3,3-alkyl-substituted) acrylic acid (3) or esters thereof in a Friedel-Crafts reaction with (alkyl-)benzenes (2), and subsequently cyclizing formed intermediate (4), polyphosphoric acid being the preferred catalyst for this cyclization.

The preparation process according to the present invention can be illustrated by the following reaction scheme:

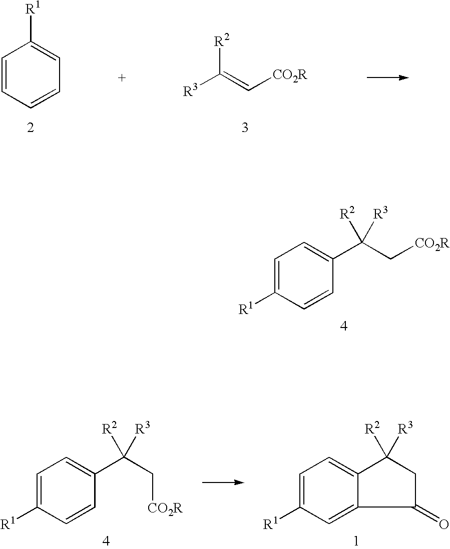

The radicals R, R¹, R² and R³ can have the following meanings:

R is hydrogen, C₁-C₄-alkyl,

R¹ is hydrogen, methyl, ethyl, n-propyl, isopropyl, n-butyl, isobutyl,

R², R³, independently of one another, are H, methyl, ethyl, n-propyl, isopropyl, n-butyl, sec-butyl, isobutyl.

Preferably, the radicals R, R¹, R² and R³ have the following meanings:

R is hydrogen, methyl, ethyl,

R¹ is methyl, ethyl, isopropyl

R², R³, independently of one another, are methyl, ethyl.

More preferably, R=H and R¹, R² and R³ are all methyl.

In the 1^{st} stage, i.e. the reaction of the (alkyl-)benzenes (2) with the acrylic acid derivative (3), Friedel-Crafts catalysts are preferably used. The Friedel-Crafts catalysts which may be used are all catalysts customary for this type of reaction. Metal halides, such as, for example, aluminum chloride, iron chloride or zinc chloride, are particularly suitable. Aluminum chloride is more preferred. The Friedel-Crafts reaction can be carried out in a wide temperature range. The Friedel-Crafts reaction is advantageously carried out at temperatures in the range from −20 to 100° C., preferably in the range from −10 to 50° C. and more preferably in the range from 0 to 25° C.

The 1^{st} stage can be carried out in the presence of nonaromatic solvents, such as, for example, ethers (e.g. methyl tert-butyl ether, tetrahydrofuran), cyclic or acyclic hydrocarbons (e.g. cyclohexane). The 1^{st} stage is preferably carried out without a solvent. Preference is given to an excess of (alkyl-)benzene (2).

The resulting intermediates (4) are of adequate purity to be reacted to give the end product (1). If required, purification can be carried out, for example by means of distillation.

The 2^{nd} stage, i.e. the cyclization of the intermediate (4) to give the 6-alkylindan-1-ones (1) according to the present invention, is preferably carried out in the presence of an acid and a water-binding agent. A preferred catalyst for the 2^{nd} stage is polyphosphoric acid. Preferred amounts are 50 to 250% by weight, based on substrate used, and preferred polyphosphoric acid contents are 110-120%, based on molar equivalents of phosphoric acid. The cyclization can advantageously be carried out at temperatures in the range from 50 to 180° C., preferably in the range from 80 to 150° C. and more preferably in the range from 100 to 130° C.

### EXAMPLES

### Example 1

### 3,3,6-Trimethylindan-1-one

a) 3-Methyl-3-(4′-methylphenyl)butyric Acid

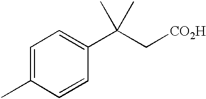

340 g (2.6 mol) of aluminum chloride were stirred at room temperature into 300 g (3.26 mol) of toluene and cooled to 0° C. A solution of 250 g (2.5 mol) of 3,3-dimethylacrylic acid in 400 g (4.34 mol) of toluene was then metered in at 0-5° C. over the course of 1 h. When the metered addition was complete, the mixture was after-stirred for a further 10 min, added to 1000 g of dilute hydrochloric acid and extracted with methyl tert-butyl ether. Distillation gave 370 g of product with a GC purity of 95%. Yield 77% of theory.

b) 3,3,6-Trimethylindan-1-one

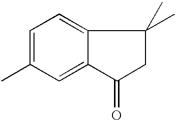

730 g of polyphosphoric acid (115% based on molar equivalents of phosphoric acid) were initially introduced and heated to 120° C. 370 g (1.93 mol) of tolylbutyric acid (crude product from the 1^{st} stage, Example 1a) were then metered in over the course of 45 min, and the mixture was after-stirred at 120° C. for a further 3 h. The mixture was cooled to 60° C. and admixed with 900 g of iced water, extracted with methyl tert-butyl ether and then distilled. 320 g of 3,3,6-trimethylindan-1-one product with a GC purity of 95% were obtained. Yield 95% of theory. APPLICATION EXAMPLES

### Example 2

### Perfume Composition 1 and 2

TABLE 2

| | Parts by weight | Parts by weight |
|---|---|---|
| Name | Composition 1 | Composition 2 |
| Red Berries extract | 40 | 40 |
| Bergamot oil bergapten-free | 80 | 80 |
| Cardamom absolute | 25 | 25 |
| Cyclopentadecanolide (1) | 190 | 190 |
| Cypress oil | 5 | 5 |
| Eugenol | 50 | 50 |
| Ginger oil synthetic | 60 | 60 |
| Methyl dihydrojasmonate | 100 | 100 |
| Iso E Super ® (2) | 200 | 200 |
| Amarocit ® (1) | 50 | 50 |
| Muscone | 50 | 50 |
| Clary sage oil | 80 | 80 |
| Stemone ® (3) | 10 | 10 |
| Vertocitral ® (1) | 50 | 50 |
| Vetiver oil | 10 | 10 |
| 3,3,6-Trimethylindan-1-one | 1 | — |

Haarmann & Reimer, Germany, Holzminden

International Flavors & Fragrances, USA, New Jersey, Union Beach Givaudan, Switzerland, Geneva

The addition of 3,3,6-trimethylindan-1-one to perfume composition 1 brings about a significant intensification of the overall odor, with the leathery notes additionally being enhanced. In addition, the perfume composition adopts a significantly sharper character, which is particularly suitable for a man's scent.

### Example 3

### Perfume Composition 3 and 4

TABLE 3

| | Parts by weight | Parts by weight |
|---|---|---|
| Name | Composition 3 | Composition 4 |
| Ambrarome abs. ® (4) | 2 | 2 |
| Benzoin resin | 100 | 100 |
| Bergamot oil bergapten-free | 12 | 12 |
| Citral | 5 | 5 |
| Civet tincture 10% in dipropylene | 20 | 20 |
| glycol | | |
| Coumarin | 19 | 19 |
| Indole 10% in dipropylene glycol | 5 | 5 |
| Isomethylionone | 30 | 30 |
| Linalool | 10 | 10 |
| Opoponax oil | 5 | 5 |
| Patchouli oil | 20 | 20 |
| Tolu balsam resin colourless | 2 | 2 |
| Vanilla resin, decolourized, | 50 | 50 |
| 10% in dipropylene glycol | | |
| 3,3,6-Trimethylindan-1-one | 5 | — |

(4) Synarome, France, Bois Colombes

The addition of 3,3,6-trimethylindan-1-one to perfume composition 3 achieves a harmonization of the overall odor in this orientally floral perfume composition. The perfume composition smells more natural and was clearly preferred over perfume composition 4 in a preference test.

### Example 4

### Perfume Composition 5 and 6

TABLE 4

| | Parts by weight | Parts by weight |
|---|---|---|
| Name | Composition 5 | Composition 6 |
| Clary sage oil synthetic | 100 | 100 |
| Davana oil | 10 | 10 |
| Benzyl salicylate | 100 | 100 |
| Undecavertol ® (3) | 5 | 5 |
| Iris oil synthetic | 5 | 5 |
| Irone | 2 | 2 |
| Coumarin | 80 | 80 |
| Nectarol ® (5) | 2 | 2 |
| Cedarwood oil Florida | 100 | 100 |
| Andrane ® (2) | 100 | 100 |
| Sclareolide | 10 | 10 |
| Ambroxide (1) | 5 | 5 |
| Isomeric mixture of equal parts of | 200 | — |
| 3,3,4-, 3,3,5-, 3,3,6- and | | |
| 3,3,7-trimethylindan-1-one | | |

Haarmann & Reimer, Germany, Holzminden

International Flavors & Fragrances, USA, New Jersey, Union Beach Givaudan, Switzerland, Geneva

(5) Firmenich, Switzerland, Geneva

The perfume composition 5 has a significantly more intensive odor in which the leathery labdanum note plays a harmonious part in the odor picture. In the case of perfume composition 6, which has a weaker odor overall, the leathery note is significantly lesser. The perfume composition is clearly more suitable for the perfuming of a care range for men.

### Example 5

### Shampoo

Perfume compositions 1 and 2 were separately incorporated, in an amount in each case of 1% by weight, into the shampoo formulation given below (Table 5), and the shampoo was used to wash hair. The odor evaluation was then carried out by a group of experts on the shampoo composition, the dilute aqueous solution in the hair, the wet rinsed hair and the dry hair.

The addition of 3,3,6-trimethylindan-1-one gives the perfume composition 1 an excellent rounded leather note compared to perfume composition 2 in all evaluation stages for the shampoo. The leathery odor is significantly perceptible particularly on wet and dry hair, and it clearly represents an improvement in the perfume composition.

TABLE 5
Shampoo formulation

| Ingredients | INCI name | % (w/w) |
|---|---|---|
| Demineralized water | Water (Aqua) | 75.45 |
| Plantacare PS 10 (1) | Sodium Laureth Sulfate (and) | 20.00 |
| | Lauryl Glycoside | |
| Sodium chloride | Sodium Chloride | 1.40 |
| Citric acid 10.0% solu- | Citric Acid | 1.65 |
| tion | | |
| Phenonip (2) | Phenoxyethanol (and) Methyl- | 0.50 |
| | paraben (and) Ethyl- | |
| | paraben (and) Propyl- | |
| | paraben (and) Butylparaben | |
| Composition 1 or 2 (3) | Fragrance | 1.00 |

Suppliers:

(1) Cognis Deutschland GmbH, D-40191 Dusseldorf, Germany

(2) Nipa Laboratories Ltd., CF382SN South Wales, UK

(3) Haarmann & Reimer, D-37603 Holzminden, Germany

### Example 6

### Washing Powder

Perfume compositions 5 and 6 were incorporated separately, in each case in an amount of 0.4% by weight, into a detergent formulation (Table 6). The odor from the powder, of the aqueous solution, of the damp and of the dry laundry was assessed by a group of experts. It was found that perfume composition 5 was generally more preferred and produced a more harmonious scent by virtue of the woody and leathery notes. On the damp and dry laundry, in particular, a significantly more intensive and typical leathery odor was perceived for perfume composition 5 compared with composition 6.

TABLE 6
Washing powder formulation

| Ingredients | INCI name | % (w/w) |
|---|---|---|
| Marlon A (1) | Sodium laurylbenzene sulphonate, | 7.0% |
| | approx. 80% | |
| Marlipal 013 (1) | C13/15-Oxoalcoholpolyglycolether | 4.0% |
| Prisavon (2) | Soybean/Coco soap, approx. | 3.5% |
| | 25% fatty acid | |
| Thylose (3) | Carboxymethylcellulose | 1.5% |
| Tinopal (4) | Optical brightener | 0.2% |
| Optimase (5) | Enzymes | 0.6% |
| Waterglass (6) | Sodium disilicate | 5.0% |
| Sodium sulfate (6) | Sodium sulfate (anhydrous) | 26.8% |
| Sodium perborate (1) | Sodium perborate (tetrahydrate) | 20.0% |
| Sodium tripolyphosphate | Sodium tripolyphosphate or | 26.0% |
| (1) | Zeolithe | |
| Magnesium silicate (6) | Magnesium silicate | 2.0% |
| TAED (3) | Bleach activator | 3.0% |
| Composition 5 or 6 (7) | Fragrance | 0.4% |

Suppliers:

Degussa-Hüls AG, D-60287 Frankfurt, Germany

Uniqema Chemie, NL-2800 M Gouda, Netherlands

Clariant GmbH, D-29699 Frankfurt, Germany

Ciba Spezialitätenchemie AG, 4000 Basel, Switzerland

Genecore Int. GmbH, D-31564 Nienburg, Germany

Merck KGaA, D-64293 Darmstadt, Germany

Haarmann & Reimer GmbH, D-37603 Holzminden, Germany

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Fragrance mixtures comprising compounds of the formula (1) 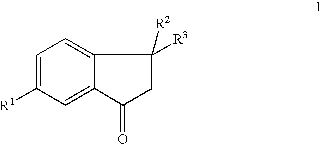
wherein
R¹ is methyl, ethyl, n-propyl, isopropyl, n-butyl, isobutyl,
R², R³, independently of one another, are H, methyl, ethyl, n-propyl, isopropyl, n-butyl, sec-butyl, isobutyl,
with the proviso that R² and R³ together contain at most 4 carbon atoms and R¹, R² and R³ together contain at most 8 carbon atoms.

2. Perfumed products comprising at least one compound of the formula (1) 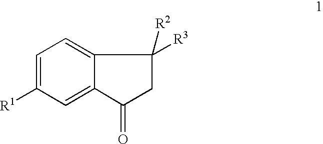
wherein
R¹ is methyl, ethyl, n-propyl, isopropyl, n-butyl, isobutyl,
R², R³, independently of one another, are H, methyl, ethyl, n-propyl, isopropyl, n-butyl, sec-butyl, isobutyl,
with the proviso that R² and R³ together contain at most 4 carbon atoms and R¹, R² and R³ together contain at most 8 carbon atoms.

3. A process for the preparation of compounds of the formula (1) 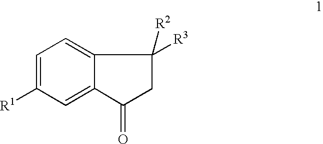
with the proviso that R² and R³ together contain at most 4 carbon atoms and R¹, R² and R³ together contain at most 8 carbon atoms, comprising the following steps:
a) reacting (alkyl-)benzenes of the formula (2) with acrylic acid derivatives of the formula (3) to give intermediates of the formula (4),
b) Cyclization of the intermediates (4) to give the compounds of the formula (1), 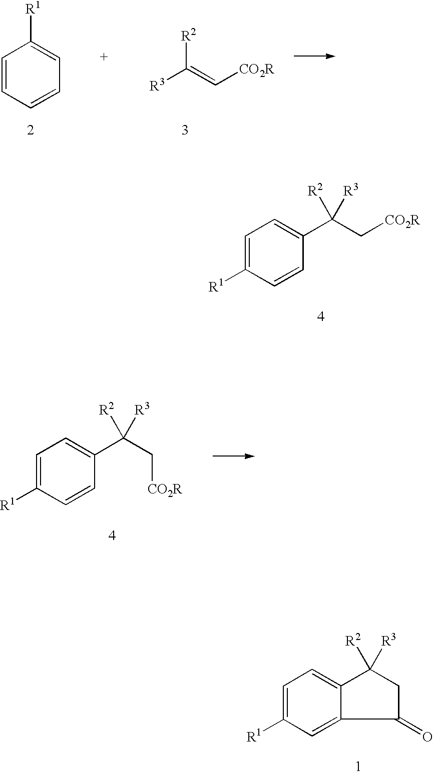
wherein the radicals R, R¹, R² and R³ have the following meanings:
R is hydrogen, C₁-C₄-alkyl,
R¹ is hydrogen, methyl, ethyl, n-propyl, isopropyl, n-butyl, isobutyl,
R², R³, independently of one another, are H, methyl, ethyl, n-propyl, isopropyl, n-butyl, sec-butyl, isobutyl.

4. A process according to claim 3, wherein step b) is carried out in the presence of polyphosphoric acid.
